(19) United States
(12) Patent Application Publication
(10) Pub. No.: US 2013/0259208 A1
(43) Pub. Date: Oct. 3, 2013

(54) RADIOGRAPHIC IMAGING APPARATUS

(71) Applicant: CANON KABUSHIKI KAISHA, Tokyo (JP)

(72) Inventor: Tetsuo Watanabe, Utsunomiya-shi (JP)

(73) Assignee: CANON KABUSHIKI KAISHA, Tokyo (JP)

(21) Appl. No.: 13/804,411

(22) Filed: Mar. 14, 2013

(30) Foreign Application Priority Data
Mar. 29, 2012 (JP) 2012-077804

(51) Int. Cl. A61B 6/00

(52) U.S. Cl. CPC A61B 6/4283
USPC 378/182; 378/167

(57) ABSTRACT

A radiographic imaging apparatus using a flexible radiation detector electrically detects radiation that was transmitted through an object to be imaged, converts the detected radiation into radiographic image information, and has a housing box that incorporates the radiation detector in an attachable and detachable manner. The housing box includes a supporting member that supports the radiation detector as a mechanical reinforcement for keeping the radiation detector flat even when under a load, and an exterior housing that contains the radiation detector and the supporting member.

### BACKGROUND OF THE INVENTION

1. Field of the Invention

The present invention relates to portable digital radiographic imaging apparatuses (electronic cassettes).

2. Description of the Related Art

Conventionally, apparatuses that obtain a radiographic image of an object by irradiating the object and detecting an intensity distribution of the radiation that was transmitted through the object are, in general, widely used in the fields of industrial non-destructive testing and medical diagnosis. For such use in imaging, apparatuses for capturing a digital radiographic image using a semiconductor sensor such as one disclosed in Japanese Patent No. 3066944 have been developed.

These systems, which have a significantly wide dynamic range and instantly obtain an output image, are becoming widespread. This kind of system is roughly divided into a radiographic imaging unit for acquiring an image and a control unit for controlling image acquisition by the imaging unit and displaying the captured image on a monitor. This apparatus uses a glass substrate as a semiconductor sensor, and is therefore supported and covered by a robust rigid body so that the apparatus is protected from damage or the like. Consequently, the weight increases, resulting in a burden on an operator.

Japanese Patent No. 4436593 proposes a radiation detector that is lightweight, and is so flexible that the apparatus can be formed to fit the surface shape of a patient.

The radiation detector disclosed in Japanese Patent No. 4436593 is independently used, rather than incorporated in a housing as in conventional apparatuses, with a focus on the flexibility of the radiation detector. For imaging that places a load on the radiation detector, a rigid body for supporting the radiation detector is needed. In other cases, for example, in the case of imaging an object to be examined on a soft bed while the doctor is making his/her rounds, the radiation detector can be distorted due to the body pressure and some kind of configuration for correcting image distortion is therefore necessary. For upright imaging in which the radiation detector needs to be disposed vertically, some kind of configuration for keeping the flexible radiation detector flat is necessary.

As described above, a problem arises in keeping the flexible radiation detector in a stable shape, without being affected by a load or posture.

The present invention provides a radiographic imaging apparatus using a flexible radiation detector that is able to perform stable imaging without being affected by a change in a load or posture.

### SUMMARY OF THE INVENTION

According to one aspect of the present invention, a radiographic imaging apparatus has the following configuration. That is, the radiographic imaging apparatus according to the present invention is a radiographic imaging apparatus using a flexible radiation detector, the apparatus electrically detecting radiation that was transmitted through an object to be imaged and converting the detected radiation into radiographic image information, comprising:

a housing box configured to incorporate the radiation detector in an attachable and detachable manner,

the housing box comprising:

a supporting member configured to support the radiation detector as a mechanical reinforcement for keeping the radiation detector flat; and

an exterior housing configured to contain the radiation detector and the supporting member.

According to the present invention, it is possible to ensure stable image quality without being affected by a change in a load or posture even with the radiographic imaging apparatus using the flexible radiation detector. In addition, with the lightweight radiographic imaging apparatus, a burden on an operator is reduced, and it is also possible to reduce outlay because existing fixtures such as stands and tables can be used.

Further features of the present invention will become apparent from the following description of exemplary embodiments (with reference to the attached drawings).

### BRIEF DESCRIPTION OF THE DRAWINGS

FIG. 1 is a cross-sectional view of an electronic cassette according to a first embodiment.

FIG. 2 is a cross-sectional view illustrating a layer structure of a detector according to the first embodiment.

FIG. 3 is a diagram illustrating a display unit of the electronic cassette according to the first embodiment.

FIG. 4 is a cross-sectional view of an electronic cassette according to a second embodiment.

FIG. 5 is a diagram illustrating a configuration in which a detector according to the second embodiment is mounted in a plurality of electronic cassettes.

FIG. 6 is a cross-sectional view of an electronic cassette according to a third embodiment.

FIG. 7 is a diagram illustrating a configuration in which a detector according to the third embodiment is mounted in an electronic cassette.

FIG. 8A is a diagram illustrating a configuration of an imaging system according to a fourth embodiment.

FIG. 8B is a diagram illustrating a configuration of an imaging system according to the fourth embodiment.

FIG. 9 is a diagram illustrating a configuration of an imaging system according to a fifth embodiment.

### DESCRIPTION OF THE EMBODIMENTS

Embodiments of the present invention will be hereinafter described in detail with reference to the drawings.

### First Embodiment

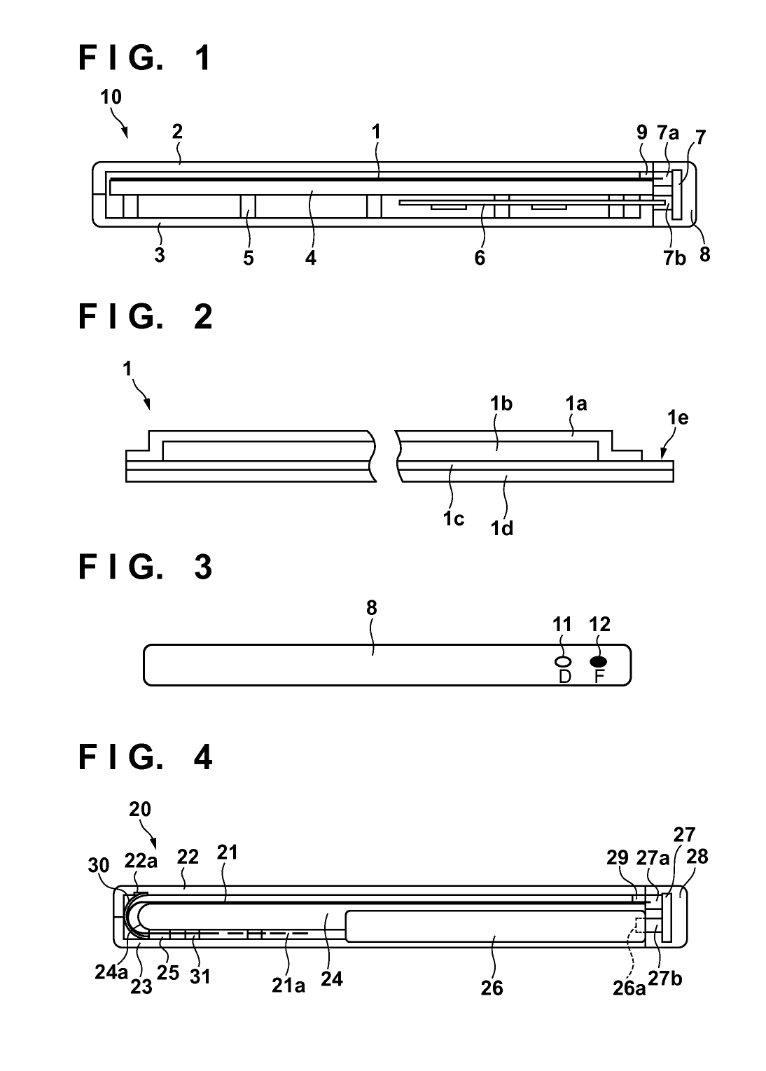

FIG. 1 is a cross-sectional view of an electronic cassette, which is a portable radiographic imaging apparatus according to a first embodiment.

In FIG. 1, numerical reference 10 denotes an electronic cassette that incorporates an x-ray image detection panel 1 and detects x-rays that were generated by an x-ray generation apparatus (not shown) and transmitted through an object to be imaged with photoelectric conversion devices (sensors) that are arrayed to form a two-dimensional lattice. An image acquired by the electronic cassette 10 is transferred to an external control device, and the acquired image is displayed on a monitor connected to the control device and used for diagnosis. FIG. 1 shows an internal cross-section of the electronic cassette 10.

The x-ray image detection panel 1, which is a radiation detector, electrically detects, as an electric signal, the x-rays that are radiation transmitted through the object, converts the electric signal into radiographic image information, and outputs it. The x-ray image detection panel 1 basically has a structure in which a fluorescent screen 1b and a photoelectric conversion device 1c are stacked so as to be sandwiched by flexible substrates 1a and 1d that respectively are front and back surfaces, as shown in FIG. 2. In many cases, a glass plate has conventionally been used as the substrate 1d, however, the first embodiment employs a resin film material, thereby achieving flexibility. Further, a control substrate 6 for performing processing for read control and electricity output in the x-ray image detection panel 1 is connected to the x-ray image detection panel 1 via an interface substrate 7.

These components are contained and used in a portable housing box in order to protect them from a load or the like deriving from the object to be examined and so that they can be readily mounted on various fixtures. The housing box roughly consists of three parts.

The first part is a supporting member 4 that functions as a mechanical reinforcement for keeping the x-ray image detection panel 1 flat even when under a load. The second part is a housing lid member 2 on an x-ray incident face (radiation incident face) side that is made of a material having a high radiolucency. The third part is a housing bottom member 3 that is fixed to the supporting member 4 via a connecting portion 5. In other words, assuming the x-ray incident face side as an upper side in a vertical (up-and-down) direction, the housing lid member 2, the supporting member 4, the connecting portion 5, and the housing bottom member 3 are configured in this order, and the housing lid member 2 and the housing bottom member 3 function as an exterior member for containing the x-ray image detection panel 1, the supporting member 4, and the connecting portion 5.

The housing lid member 2, the supporting member 4, the connecting portion 5, and the housing bottom member 3 are combined with one another to form an opening 9 into which the x-ray image detection panel 1 is inserted from the side and a narrow gap in an incidence direction for housing the x-ray image detection panel 1. On the side of the side face with the opening 9, a side face cover member 8 that contains the interface substrate 7 is provided so as to be attachable to and detachable from the housing lid member 2 and the housing bottom member 3. In a state where the side face cover member 8 is attached, a housing box is formed that is sealed so that external light does not enter.

Connectors 7a and 7b on the interface substrate 7 electrically connect to the x-ray image detection panel 1 and the control substrate 6, respectively. First, the x-ray image detection panel 1 is put into the connector 7a at a contact portion 1e shown in FIG. 2, and is inserted into the inner gap from the opening 9 in the connected state. Lastly, the control substrate 6 is connected to the connector 7b, the opening 9 is covered and sealed with the side face cover member 8, and the electronic cassette 10 serving as the x-ray imaging apparatus is thereby configured. Unlike conventional x-ray image detection panels that use a glass plate as a substrate, with the above-described structure, specifications required for the strength can be made small, and it is therefore possible to achieve a comprehensive reduction in the weight and employ an appropriate weight with which portability is attained.

The configuration of the thus sealed housing box also enables a seat material such as a film or photostimulable phosphor to be mounted. Even if the electronic cassette 10 fails for some reason, imaging can be performed alternatively with a film or photostimulable phosphor, which shortens the time period during which imaging cannot be performed. When such an operation is carried out, a configuration is desired for recognizing what kind of medium is mounted (housed) in the housing box.

The electronic cassette 10 according to the first embodiment is provided with a detection unit that detects whether or not the x-ray image detection panel 1 is mounted (whether or not the x-ray image detection panel 1 is housed), and with a display unit with which the mounted x-ray image detection panel 1 can be visually checked on the surface of the side face cover member 8. For example, FIG. 3 shows the surface of the side face cover member 8, on which display windows 11 and 12 are configured as the display unit. The display windows 11 and 12 are defined respectively by “D”, which indicates digital radiography, and “F”, which indicates a sheet material such as a film or photostimulable phosphor that is not an electronic material.

A slidable indicator is provided inside the display windows 11 and 12, whose color of display is changed by the slidable indicator being placed at the position of the display window 11 or 12. Usually, the display on the display window 12 side, that is, the display of “F” is colored, while when the x-ray image detection panel 1 is mounted to the connector 7a, the indicator is moved in conjunction therewith by a slide mechanism (not shown), and the display of “D” on the display window 11 side is colored. Thus, the display windows 11 and 12 function as a notification unit that indicates whether or not the x-ray image detection panel 1 is housed in the housing box.

Note that a configuration can also be easily implemented in which an indicator such as an LED, which serves as an electric indicator in place of the aforementioned mechanical mechanism, is illuminated upon connection of the x-ray image detection panel 1 being electrically detected. If such electrical detection is possible, it is also possible to switch irradiation conditions and image processing conditions of the x-ray generation apparatus to the settings suitable for the x-ray image detection panel 1 by transmitting the detection result to the external control device. Further, comprehensive outlay can be reduced because existing fixtures can be used by forming the outer shape of the aforementioned electronic cassette 10 in the same shape as existing cassettes.

As described above, according to the first embodiment, the housing box that incorporates the x-ray image detection panel, which is a flexible radiation detector, in an attachable and detachable manner is constituted by the supporting member that supports the x-ray image detection panel as a mechanical reinforcement, and the exterior housing. It is thus possible to achieve stable imaging without being affected by a change in a load or posture even with a radiographic imaging apparatus using a flexible radiation detector.

### Second Embodiment

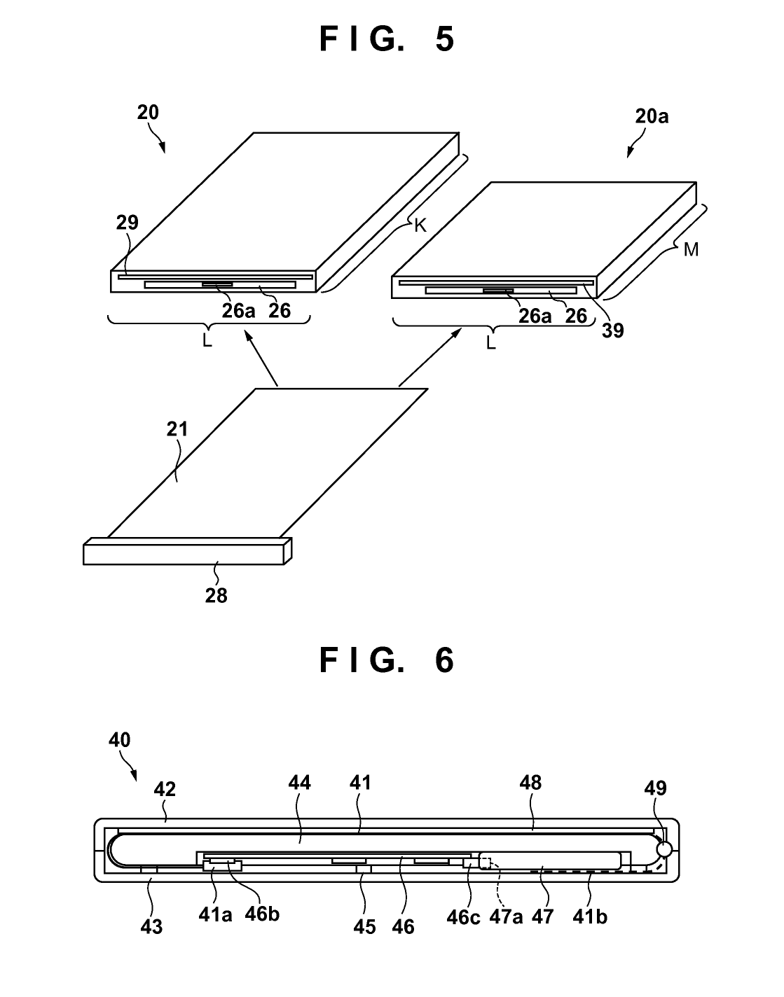

FIG. 4 is a cross-sectional view of an electronic cassette according to a second embodiment, and FIG. 5 is a diagram showing a state where a detector according to the second embodiment is mounted in electronic cassettes with different dimensions.

An x-ray image detection panel 21 shown in FIG. 4 is folded and housed within a housing box, using its flexibility. The housing box is formed with a supporting member 24 that functions as a mechanical reinforcement for keeping the x-ray image detection panel 21 flat even when under a load, as in the first embodiment, a housing lid member 22, a housing bottom member 23, a connecting member 25, and so on.

In addition, a side face cover member 28 is provided for sealing an opening 29 of the x-ray image detection panel 21, and a control substrate 27 for controlling an imaging operation of the x-ray image detection panel 21 and acquiring output is disposed inside. The control substrate 27 is connected to the x-ray image detection panel 21 through a connector 27a, and is connected to a battery unit 26 that is disposed within the housing box through a connector 27b and supplies electricity. The x-ray image detection panel 21 connected to the control substrate 27 is inserted into an inner gap from the opening 29, and is connected to the battery unit 26 and the connector 27b. Lastly, the opening 29 is covered and sealed with the side face cover member 28, and the electronic cassette 20 serving as the x-ray imaging apparatus is thereby configured.

At an inner end that is opposed to the opening 29 (an inner end of the exterior housing), a guide plate 30 is provided for guiding a leading edge of the x-ray image detection panel 21 in a folding direction (a housing direction in which the x-ray image detection panel 21 is housed within the housing box). The guide plate 30 is made of a highly slidable material, and is formed in a state of having a curvature to form a fixed gap from a tip portion 24a in a curved shape of the supporting member 24. In addition, an end of the guide plate 30 is embedded in a groove portion 22a formed on the housing lid member 22 so as not to impede guiding of the x-ray image detection panel 21 when inserted. With such a guide path, the x-ray image detection panel 21 is folded downward from its incident side and back under its back side when housed.

The amount of folding, which is a state of housing, is different depending on the length of the x-ray image detection panel 21, while inside the housing box, an electric contact portion 31 is formed that comes in contact with the x-ray image detection panel 21 so as not to have a large resistance thereto in the insertion direction. On the x-ray image detection panel 21, a detection circuit (detection unit) is formed that is connected to the electric contact portion 31 outside an effective imaging area and detects the folding amount. In the case where the x-ray image detection panel 21 has a folded portion 21a indicated with a broken line in the diagram and in the state of folding indicated by a solid line, the folding amount is detected based on a difference in wiring resistance of the detection circuit. The size of the effective area that works in a state of being housed in the housing box is calculated based on information regarding the detected folding amount and an effective imaging area that is known in advance, and when imaging is performed, only pixels in this effective area are driven to acquire an image. Thus, excessive power consumption is suppressed, and unnecessary processing such as cutting of a needless portion at the time of image generation does not have to be performed.

Electronic cassettes having different field-of-view dimensions are sought in accordance with the imaging mode. When using conventional films or photostimulable phosphor, which are consumed materials and inexpensive, it has been relatively easy to prepare those with different dimensions. However, in the case of electronic cassettes, which cost more, it is more difficult to prepare those with different dimensions in terms of costs. On the contrary, with the configuration in which the x-ray image detection panel 21 is folded and housed in the housing box as in the second embodiment, it is possible to use a single x-ray image detection panel in housing boxes having different effective imaging areas.

In other words, it is possible to use the same single x-ray image detection panel in a plurality of housing boxes with different dimensions that have a common dimension of a side to be a line of intersection between a side face of each housing box on which the opening is formed and its upper face on which x-rays toward the housing box are incident, and have different dimensions of the other side of the upper face.

In an electronic cassette 20 shown in FIG. 5, the dimension of the outer planar shape (rectangle) viewed from its x-ray incident face side is L×K, and the opening 29 that functions as an insertion slot into which the x-ray image detection panel 21 is inserted is formed on the side face on the dimension L side. Meanwhile, the battery unit 26 is mounted in a state of exposing a connecting terminal 26a. The side face cover member 28 that contains the control substrate is mounted in the battery unit 26, and is thus connected to the x-ray image detection panel 21 and the battery unit 26.

On the other hand, in an electronic cassette 20a having different dimensions from those of the electronic cassette 20, the dimension of the outer planar shape (rectangle) viewed from its x-ray incident face side is L×M, and thus, the electronic cassette 20a has an outer shape in which the dimension L in one direction is the same as that of the electronic cassette 20. The shape on the side of the face having the same dimension is also the same as that of the electronic cassette 20, and the dimensions of an opening 39 and the battery unit 26 are also the same. Therefore, the x-ray image detection panel 21 and the side face cover member 28 can be used in both the electronic cassette 20 and the electronic cassette 20a.

The difference is the folding state (housing state) of the x-ray image detection panel 21 within the housing box, and the folding amount in the electronic cassette 20a is larger than that in the electronic cassette 20 by K−M. With the detection circuit for detecting the folding amount shown in FIG. 4, appropriate effective imaging areas can be set in the electronic cassettes 20 and 20a.

As described above, according to the second embodiment, the x-ray image detection panel can be folded and housed within housing boxes with various dimensions using the flexibility of the x-ray image detection panel, thereby enabling an expensive x-ray image detection panel portion to be used in different housing boxes, and it is thus possible to reduce comprehensive outlay. For example, many kinds of imaging techniques can be covered if the field-of-view dimension of the electronic cassette is set to 43 cm×35 cm, which is known as half size, and the dimension of the electronic cassette itself is set to 35 cm×28 cm, which is known as large quarter size. In addition, a battery can also be used in different housing boxes by employing a common specification, and it is thereby possible to improve efficiency in use and easily replace the battery when it is not sufficiently charged.

### Third Embodiment

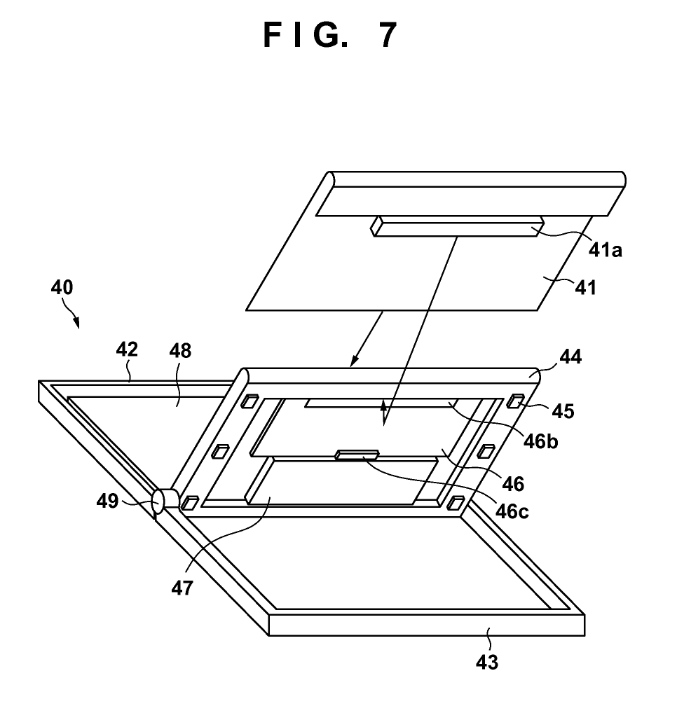

FIG. 6 is a cross-sectional view of an electronic cassette according to a third embodiment. The electronic cassette 40 according to the third embodiment is an example in which the mode of its insertion into the housing box is different from that in the first and second embodiments, and the mode of folding is also different from that in the second embodiment. FIG. 6 is a cross-sectional view showing a state where an x-ray image detection panel 41 is mounted, and FIG. 7 is a perspective view showing how the x-ray image detection panel 41 is mounted.

The point that the housing box roughly consists of three parts, namely a supporting member 44, a housing lid member 42, and a housing bottom member 43 that is fixed to the supporting member 44 via a connecting portion 45, is the same as in the above-described embodiments. According to the fourth embodiment, these three members are joined to a hinge 49, and the housing box has a structure capable of being pivotably opened and closed, which is the different configuration from the other embodiments. A dent portion is formed on the back face that is opposed to an incident face of the supporting member 44, and a control substrate 46 for controlling an imaging operation of the x-ray image detection panel 41 and acquiring output and a battery 47 for supplying electricity are disposed. The control substrate 46 and the battery 47 are electrically connected to each other via connectors 46c and 47a.

A connector 41a is provided at an end of the x-ray image detection panel 41, and is connected to a connector 46b formed on the control substrate 46. Both ends of the supporting member 44 have a curved shape, the x-ray image detection panel 41 connected to the connector 46b is disposed along the surface of the supporting member 44, and the other end of the x-ray image detection panel 41 that is opposed to the connector 41a is drawn onto the hinge 49 side. If the longitudinal side of the x-ray image detection panel 41 is longer than the corresponding side of the supporting member 44, the x-ray image detection panel 41 is mounted so as to embrace the supporting member 44 up to the back side thereof, as the folded portion 41b indicated by a broken line. Since the effective imaging area to be dealt with by the control substrate 46 is set therein in advance, pixels disposed from the connector 41a side up to a fixed distance are driven as the effective area. Accordingly, such a portion as the folded portion 41b that extends to reach the back face when housed is not driven because the distance from the connector 41a is larger than a set value. Therefore, even if the detection circuit for detecting the folding amount (length) as that in the second embodiment is not provided, a similar function can be achieved.

A buffer member 48 is provided between the incident face side of the supporting member 44 and the inner wall of the housing lid member 42. The buffer member 48, with which the inner wall of the housing lid member 42 and the x-ray image detection panel 41 locally come in contact with each other when a load is applied on the incident face side, has an effect of preventing an increase in pressure and dispersing the pressure. In addition, the buffer member 48 has another effect of keeping the shape of the x-ray image detection panel 41 flat by pressing the x-ray image detection panel 41 against the supporting member 44 side with the elasticity of the buffer member 48. Further, the incident face of the housing box is not divided by a side face cover member as in the first and second embodiments, but is formed with a single member, which is appropriate for keeping a clean state by cleaning.

When mounting, the supporting member 44, the housing lid member 42, and the housing bottom member 43 are opened around the hinge 49 as shown in FIG. 7, and closed again after the x-ray image detection panel 41 is attached to the supporting member 44, thereby configuring the electronic cassette 40. As described above, the x-ray image detection panel is simply sandwiched, rather than being inserted from the side as in the first and second embodiments, and it is therefore possible to suppress damage of the x-ray image detection panel caused by sliding.

### Fourth Embodiment

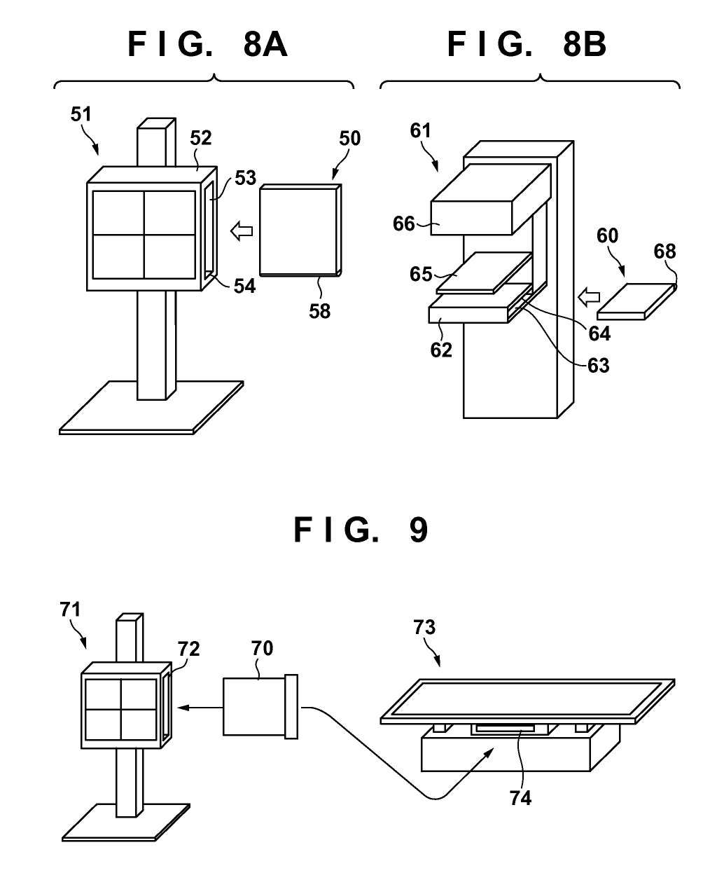

When an x-ray image is actually captured, it is necessary to change the positional relationship between an electronic cassette and an object to be examined in accordance with the position to be imaged or the state of the object. Therefore, various fixtures are used in order to keep a constant state of the electronic cassette that is positioned relative to the object. Depending on imaging techniques, the effective imaging area of the x-ray image detection panel needs to be brought closer to the object body. Examples include a stand 51 used in upright imaging of the chest or the like shown in FIG. 8A, and a mammography imaging apparatus 61 shown in FIG. 8B.

The stand 51 is a device for chest imaging with which the object puts his/her chin on the upper part of a holder 52 and brings his/her chest in close contact with the front face of the holder 52. Accordingly, a wide range that covers up to the upper part of the thorax is imaged in the case of the stand 51, and it is therefore important to set a short distance from the chin with which the electronic cassette is in contact to the effective imaging area.

On the other hand, the mammography imaging apparatus 61 is an apparatus for performing imaging by sandwiching and holding the breast together with a holder 62 that houses a cassette, using a compression plate 65, and performing x-ray radiation from a tube 66 from above. In this case, in order to widely image the breast, it is necessary to set a short distance from the chest wall with which the holder 62 is in contact to the effective imaging area. In the electronic cassette 10 described in the first embodiment, the outer shape of the electronic cassette and the distance to the effective imaging area are different at the left and right ends in FIG. 1. On the right side where the side face cover member 8 is provided, the distance is larger because an electronic substrate and the like are incorporated, while nothing is mounted on the opposite left side, and the distance to the effective imaging area can be approximately the same as the distance to the housing wall.

Accordingly, in the aforementioned stand 51 and mammography imaging apparatus 61, an arrangement is desired in which the end side of the electronic cassette 10 that is opposite to the side face cover member 8 is always disposed on the object side. In the electronic cassettes 50 and 60 according to the fourth embodiment, the side face cover members 58 and 68 have a shape with a thickness that is slightly thinner than that of the respective housing boxes. The electronic cassettes 50 and 60, when incorporated in the fixtures, are mounted respectively from an opening 53 that is open to the side face of the stand 51 and an opening 63 that is open to the side face of the holder 62 in the mammography imaging apparatus 61, and used for imaging. The openings 53 and 63 of the insertion paths in the fixtures are provided with guide plates 54 and 64, respectively, on one end. With this configuration, the insertion orientation of each electronic cassette is limited, and the electronic cassette can be always mounted (fixed) in a direction appropriate for the fixture. In other words, a fixture is configured that has a holder into which the x-ray image detection panel is inserted in an orientation in which the folding side of the x-ray image detection panel comes close to the object side.

Note that although an example of mounting each electronic cassette in a fixture is given in the fourth embodiment, it is desired that the electronic cassette be independently used to make effective use of the flexibility for a stretcher having a portion with a curved surface on which the object is placed. Each electronic cassette can be effectively used in accordance with the imaging mode.

### Fifth Embodiment

A fifth embodiment is an example of imaging systems in which portions for housing electronic cassettes in an upright stand 71 and an imaging stage 73 have the same configuration as that of the above-described electronic cassette, and in which a flexible x-ray image detection panel 70 itself can be directly attached to and detached for replacement. FIG. 9 shows exemplary imaging systems that are operated by exchanging the x-ray image detection panel 70 between the upright stand 71 and the imaging stage 73 that is used to image the chest, abdomen, or the like of an object in a lying position in which the object lies horizontally.

Although the fifth embodiment is the case of employing the same electronic cassette configuration as that in the first and second embodiments in which the electronic cassette is inserted from the side, the method of opening and closing the electronic cassette with a hinge as in the third embodiment can also be employed. Thus, an operation is possible in which the housing box for the electronic cassette is eliminated by employing the fixture in the mode fitted to the flexible x-ray image detection panel.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-077804, filed on Mar. 29, 2012 which is hereby incorporated by reference herein in its entirety.

What is claimed is:

1. A radiographic imaging apparatus using a flexible radiation detector, the apparatus electrically detecting radiation that was transmitted through an object to be imaged and converting the detected radiation into radiographic image information, comprising:
a housing box configured to incorporate the radiation detector in an attachable and detachable manner,
the housing box comprising:
a supporting member configured to support the radiation detector as a mechanical reinforcement for keeping the radiation detector flat; and
an exterior housing configured to contain the radiation detector and the supporting member.

2. The radiographic imaging apparatus according to claim 1,
wherein the radiation detector is housed in the housing box while being folded at an end of the radiation detector.

3. The radiographic imaging apparatus according to claim 1,
wherein the housing box further comprises:
an opening into which the radiation detector is inserted from a side of the housing box;
a guide plate that is provided at an inner end of the exterior housing which is opposed to the opening, and that is configured to guide a leading edge of the radiation detector in a folding direction upon the radiation detector being inserted, the guide plate being formed in a state of having a curvature to form a fixed gap from a tip portion in a curved shape of the supporting member; and
a guide path configured to guide the leading edge of the radiation detector that is folded by the guide plate,
wherein the radiation detector, when housed, is folded from the leading edge thereof by the guide plate and the guide path upon being inserted from the opening into the housing box.

4. The radiographic imaging apparatus according to claim 3,
wherein the housing box comprises a plurality of housing boxes with different dimensions which are provided that have a common dimension of a side to be a line of intersection between a side face of the housing box on which the opening is formed and an upper face of the housing on which radiation toward the housing box is incident, and have different dimensions of the other side of the upper face, and
the single radiation detector can be used in the plurality of housing boxes with the different dimensions.

5. The radiographic imaging apparatus according to claim 1,
wherein the housing box has a detection unit configured to detect whether or not the radiation detector is housed.

6. The radiographic imaging apparatus according to claim 5,
wherein the housing box has a notification unit configured to indicate whether or not the radiation detector is housed, using the detection unit.

7. The radiographic imaging apparatus according to claim 5,
wherein the detection unit is further configured to detect an amount of folding at the leading edge of the radiation detector as a housing state of the radiation detector, and set an effective imaging area of the radiation detector based on the detected amount.

8. The radiographic imaging apparatus according to claim 1,
wherein the housing box further incorporates a battery unit.

9. The radiographic imaging apparatus according to claim 3,
further comprising a fixture having a holder into which the radiation detector is inserted in an orientation in which a folded side of the radiation detector comes close to the object to be imaged.

10. The radiographic imaging apparatus according to claim 1,
wherein the housing box is a portable housing box.

11. The radiographic imaging apparatus according to claim 1,
wherein the housing box is fixed to a fixture.

12. The radiographic imaging apparatus according to claim 1,
wherein the housing box is able to house a sheet material that includes at least one of a film or photostimulable phosphor.
